(19) United States
(12) Patent Application Publication
(10) Pub. No.: US 2024/0366706 A1
(43) Pub. Date: Nov. 7, 2024

(54) USE OF CISTANCHE TUBULOSA EXTRACT IN THE PREPARATION OF A MEDICAMENT FOR RELIEVING DRY EYE SYNDROME

(71) Applicant: SINPHAR TIAN-LI PHARMACEUTICAL CO., LTD. (HANGZHOU), Hangzhou, Zhejiang (CN)

(72) Inventors: Han-peng KUO, Chung Shan, Taiwan (CN); Yao-yi CHENG, Chung Shan, Taiwan (CN)

(21) Appl. No.: 18/575,018

(22) PCT Filed: May 6, 2022
(86) PCT No.: PCT/CN2022/091159

§ 371 Date: Dec. 28, 2023

Related U.S. Application Data
(60) Provisional application No. 63/185,320, filed on May 6, 2021.
(60) Provisional application No. 63/244,129, filed on Sep. 14, 2021.

(51) Int. Cl. A61K 36/64; A61K 9/00; A61K 31/047; A61K 31/202; A61K 31/355; A61K 31/4415; A61K 36/185; A61P 27/02

(52) U.S. Cl. CPC A61K 36/64; A61K 9/0053; A61K 31/047; A61K 31/202; A61K 31/355; A61K 31/4415; A61K 36/185; A61P 27/02

(57) ABSTRACT

Use of Cistanche tubulosa extract in preparation of a medicament for relieving dry eye symptoms. Said symptoms include ocular burning, itching, stinging, excess watering, foreign body sensation, pain, redness, photophobia and blurred vision.

### TECHNICAL FIELD

The present disclosure provides a method for relieving dry eye syndrome by administering an effective amount of Cistanche tubulosa extract.

### BACKGROUND

Dry eye syndrome, also known as keratoconjunctive sicca, is one of common eye diseases nowadays, characterized by a reduction in the quantity or quality of tears. Without proper management, dry eye syndrome may lead to corneal ulceration or permanent damage to visual acuity. Dry eye syndrome is often associated with environmental factors such as allergens, smoke, particulates, dry air and air conditioning. Other factors include aging, hormone changes, blepharitis, Sjögren's syndrome, lupus, medication, prolonged use of electronic devices and contact lens wear. Refractive surgery such as laser-assisted in situ keratomileusis (LASIK) that permanently changes the shape of the cornea can also result in dry eye syndrome. Balanced tear production and elimination is vital for tear film integrity, stability and normal osmolality. Imbalances cause alterations of the tear film structure and composition, ultimately leading to tear film instability and measurable tear film hyperosmolality. Elevated tear film osmolality is considered a core mechanism in dry eye, forming the basis of dry eye symptoms and leading to ocular surface damage (Clin Exp Optom 2012; 95: 1: 3-11). Moreover, inflammation is known as an underlying cause of dry eye syndrome, with cytokine release involved.

Tear substitutes have been used to provide palliative relief to dry eye syndrome. As the effect of tear substitutes is temporary, multiple instillations throughout a day are required. Corticosteroid or cyclosporine eye drop formulations have been used to relieve dry eye. However, active ingredients, excipients or preservatives in eye drop formulations may adversely affect the ocular surface, and long-term use should be avoided. Therefore, there is a need to develop a method for better managing dry eye.

### SUMMARY

The present disclosure provides a method for relieving dry eye syndrome comprising administering to a subject in need thereof an effective amount of Cistanche tubulosa extract.

In certain embodiments, the present disclosure provides a method for increasing tear production, improving meibomian gland dysfunction, increasing tear film break-up time or increasing tear film lipid layer thickness comprising administering to a subject in need thereof an effective amount of Cistanche tubulosa extract.

In certain embodiments, the present disclosure provides a method for relieving dry eye syndrome associated with smoke, particulates, low humidity, air conditioning, prolonged use of electronic devices or contact lens wear.

In certain embodiments, the present disclosure provides a method for relieving dry eye syndrome associated with age, inflammation, autoimmune disease, refractive surgery or medication.

In some embodiments, the present disclosure provides a method for relieving dry eye syndrome comprising administering Cistanche tubulosa extract in combination with additional dietary supplements.

In another aspect, the present disclosure provides a dietary supplement for relieving eye discomfort or dry eye comprising Cistanche tubulosa extract.

### BRIEF DESCRIPTION OF THE DRAWINGS

FIG. 1 Cytotoxicity of (a) 0.03% DMSO, and (b) ST01 in corneal cell line 2.040 pRSV-T for 24 h by MTT assay (n=2). Significance was defined as follows: *P<0.05; *P<0.01, compared with control group.

FIG. 2 Effect of ST01 on hyperosmolarity-induced cell death in corneal cell line 2.040 pRSV-T for 24 h by MTT assay (n=4). Significance was defined as follows: *P<0.05; **P<0.01, compared with control group.

FIG. 3 Effects of ST01 on hyperosmolar stress-stimulated secretion of (a) TNF-α and (b) IFN-γ by corneal cell line 2.040 pRSV-T for 24 h (n=4). Significance was defined as follows: *P<0.05; **P<0.01, compared with control group.

### DETAILED DESCRIPTION OF PREFERRED EMBODIMENTS

As used herein, the term “subject” refers to a mammal, for example a human or an animal. In embodiments, the subject is suffering from dry eye syndrome. In particular embodiments, symptoms of dry eye syndrome include but not limited to a dry, scratchy, gritty, or sandy feeling in the eye, foreign body sensation, pain or soreness, stinging or burning, itching, increased blinking, eye fatigue, photophobia, blurry vision and redness. Not all subjects suffering from dry eye syndrome exhibit all symptoms simultaneously.

As used herein, the term “relieve” is to describe a process by which the severity of a sign or symptom of a disorder decreases. A sign or symptom can be alleviated without being removed. In a preferred embodiment, the administration of Cistanche tubulosa extract leads to the removal of a sign or symptom, however, removal is not necessary. Effective doses are expected to decrease the severity of a sign or symptom.

The term “effective amount” is an amount sufficient to achieve beneficial or desired results. Particularly, it is an amount sufficient for slowing down or stopping the progression, aggravation, or deterioration of one or more symptoms of a disorder or condition; or relieving one or more symptoms of a disorder or condition. Typically, amount-effect relationships from in vitro and/or in vivo tests initially can provide useful guidance on the proper amount for human body.

The term “dietary supplement” refers to a product that contains a vitamin, mineral, herb or other botanical, amino acid, fatty acid, extract, or combinations of these ingredients.

The present disclosure provides a method for relieving dry eye syndrome with Cistanche tubulosa extract. Dry eye syndrome is a multifactorial and complex disorders involving several factors such as tear hyperosmolarity, tear film instability, abnormalities of the tear film lipid layer and meibomian gland dysfunction. Hyperosmolar tear fluid may activate an inflammatory cascade and cause the release of inflammatory mediators into the tear fluid, with multiple pathophysiological effects eventually leading to increased tear film evaporation and tear film instability.

Hyperosmolar tear fluid may result from excessive tear film evaporation. Accordingly, in certain embodiments, the method provided herein increases tear production, tear film break-up time or tear film lipid layer thickness with an effective amount of Cistanche tubulosa extract.

In certain embodiments, the method provided herein improves meibomian gland dysfunction with an effective amount of Cistanche tubulosa extract.

In some embodiments, dry eye syndrome is associated with environmental factors including but not limited to allergens, smoke, particulates, dry air and air conditioning.

In some embodiments, dry eye syndrome is associated with life style including but not limited to prolonged use of electronic devices and contact lens wear.

Tear function may be compromised with age, hormonal changes due to pregnancy, contraceptive use or menopause and surgeries of cataract, photorefractive keratectomy (PRK) and LASIK. In some embodiments of the present disclosure, dry eye syndrome is associated with changes in age, hormone and ocular surface.

In some embodiments, dry eye syndrome is associated with medical conditions including but not limited to inflammation of the eyelids (blepharitis), inflammation of the surfaces of the eye, the inward or outward turning of eyelids, and autoimmune diseases such as Sjogren's syndrome, rheumatoid arthritis, and lupus erythematosus.

In some embodiments, dry eye syndrome is associated with medicines including but not limited to antihistamines, decongestants, blood pressure medications, and antidepressants.

In certain embodiments, the present disclosure provides a dietary supplement for relieving eye discomfort or dry eye comprising Cistanche tubulosa extract.

Symptoms of dry eye syndrome varies. Schirmer's test, corneal staining, dry eye questionnaire, Tear Film Break-Up Time (TFBUT) and Ocular Surface Analyzer (O. S.A) are known as tests for severity of or improvements in dry eye. Dry eye questionnaire can be a known questionnaire such as Standard Patient Evaluation of Eye (SPEED) and Ocular Surface Disease Index (OSDI) or self-developed questionnaire based on practice needs.

Ocular Surface Disease Index (OSDI) is a 12-item questionnaire designed to provide a rapid assessment of the symptoms of dry eye syndrome and their impact on vision-related function. Subjects are asked to evaluate the frequency of various symptoms, related visual functions, and environmental triggers of dry eye using a Likert-type scale ranging from 0 to 4 points ((0=none of the time; 1=some of the time; 2=half of the time; 3=most of the time; 4=all of the time). This is evaluated overall, not per eye. (Archives of Ophthalmology. 2000; 118(5):615-621; Ocular Immunology and Inflammation, 15:389-393, 2007)

OSDI includes three question types:

“Have you experienced any of the following during the last week?”
1. Eyes that are sensitive to light?
2. Eyes that feel gritty?
3. Painful or sore eyes?
4. Blurred vision?
5. Poor vision?
   “Have problems with your eyes limited you in performing any of the following during the last week?”
6. Reading?
7. Driving at night?
8. Working with a computer or a bank machine (ATM)?
9. Watching TV?
   “Have your eyes felt uncomfortable in any of the following situations during the last week?”
10. Windy condition?
11. Places or areas with low humidity?
12. Areas that are air conditioned?

Ocular Surface Analyzer (O.S.A) is known as a non-invasive dry eye analyzer. Specifically, lipid quantity and tear film lipid layer thickness are assessed by interferometry, images of both the upper and lower eyelid are analyzed by infrared meibography, time between the last complete blink and the appearance of the first discontinuity of the tear film in seconds is measured (non-invasive break-up time) and tear volume is estimated by measuring tear meniscus height.

Herba Cistanche was originally recorded in the Classic of Herbal Medicine (“Shennong Bencao Jing” in Chinese), a well-known book on medicinal plants written between about 200 and 250 AD. In traditional Chinese medicine, Herba Cistanche is frequently used to treat chronic renal disease, impotence, female infertility, morbid leucorrhea, profuse metrorrhagia, and senile constipation. Cistanche tubulosa is one of twenty-two Cistanche species in holoparasitic desert genus. It is unexpectedly found that Cistanche tubulosa extract can protect corneal epithelial cells from damages induced by hyperosmolarity which is a core mechanism in dry eye. Accordingly, Cistanche tubulosa extract can relieve symptoms of dry eye syndrome including but not limited to ocular burning, itching, stinging, excess watering, foreign body sensation, pain, redness, photophobia and blurred vision.

In certain embodiments, Cistanche tubulosa extract can be prepared as described in U.S. Pat. No. 7,087,252 hereby incorporated by reference in their entirety. Preferably, Cistanche tubulosa extract comprises echinacocide, aceteoside, isoacteoside, and tubuloside A.

Cistanche tubulosa extract can be formulated, alone or together, in suitable dosage unit formulations containing pharmaceutically acceptable carriers, adjuvants, excipients, and vehicles appropriate for each route of administration. In certain embodiments, Cistanche tubulosa extract is administered orally in a pharmaceutically acceptable formulation selected from the group consisting of a pill, tablet, capsule, powder, granule, non-aqueous liquid, aqueous liquid, suspension, solution, emulsion, and lyophilized formulation.

In the methods described herein, Cistanche tubulosa extract is administered in an amount of 50, 75, 100, 125, 150, 175, 200, 225 or 250 mg once daily.

In some embodiments, the present disclosure provides a method for relieving dry eye syndrome comprising administering Cistanche tubulosa extract in combination with additional dietary supplements. The dietary supplements include but are not limited to docosahexaenoic acid, eicosapentanoic acid, lactoferrin, linolenic acid, lutein, maqui berry extract, phosphatidylethanolamine, phosphatidylserine, phytosterols, sphingomyelin, turmeric extract, vitamin B6, vitamin C and vitamin E. Cistanche tubulosa extract and additional dietary supplements work synergistically to help relieve dry eye.

In certain embodiments, Cistanche tubulosa extract can be co-administered with lutein. Cistanche tubulosa extract and lutein can be administered in a simultaneous, sequential or alternating manner. In some embodiments, lutein can be administered at an amount between 6 mg to 30 mg.

### EXAMPLES

### Example 1: Preparation of Cistanche tubulosa Extract

10 kg of fleshy stem of Cistanche tubulosa was sliced and soaked in water which was 8 times the volume of the flakes. The flakes were soaked for 1 hour, decocted for 2 hours and filtered to obtain a first filtrate. The residue was then decocted in water 6 times the volume for 1 hour and then filtered to obtain a second filtrate. A third filtrate and a fourth filtrate were also obtained by the same procedures as the second filtrate. The four filtrates were combined, concentrated under reduced pressure at 50° C. to a specific gravity of 1.10. The concentrated filtrate was mixed with ethanol to form a mixture of 60% ethanol, which was then refrigerated for 12 hours. Thereafter, a supernatant was harvested from the cooled mixture while the residue was filtered to obtain a filtrate, which was combined with the supernatant to form a crude extract. The crude extract was concentrated in vacuo at 50° C. to have a specific gravity of 1.10, in which the ethanol was recycled. The crude extract produced has a weight of 6 kg.

The crude extract was added to water of 1 time the volume, and the mixture was heated to dissolve the crude extract. The resulting solution was extracted by macroporous adsorption resin column. Thereafter, the column was washed with water of 4 times the volume of the column first and 40% ethanol of 5 times the volume of the column second. The water eluent was extracted by the macroporous adsorption resin column again. Then the column was washed with water of 3 times the volume of the column first and 40% ethanol of 4 times the volume of the column second. The resulting water eluate was discarded. Two 40% ethanol eluates were combined and concentrated until dryness to obtain about 1.1 kg of Cistanche tubulosa extract.

### Example 2: Hyperosmolarity-Induced Damage in Human Corneal Epithelium Cell

### Methods

### Human Corneal Epithelial Cell Proliferation

### Materials

Culture medium: Keratinocyte-serum free medium (ThermoFisher 17005-042) was mixed thoroughly with 5 ng/mL human recombinant EGF, 0.05 mg/mL bovine pituitary extract, 0.005 mg/mL insulin and 500 ng/mL hydrocortisone.

Coating solution of culture plate: 0.01 mg/mL fibronectin, 0.03 mg/mL bovine collagen type I and 0.01 mg/mL bovine serum albumin (BSA) were mixed thoroughly.

MTT solution (5 mg/mL): 10 mg MTT was dissolved in 2 mL DPBS.

### Process

When coating a flask with a growth area of 75 cm², 4.5 mL of the Fibronectin/BSA/Collagen solution was added and incubated at CO₂ level of 5% and 37° C. for 24 hours. Cells were taken from working stock cell bank and warmed up to 37° C. quickly to avoid cell damages by ice crystals. Then the coating solution was removed from the flask. After cells were seeded, cell medium was added slowly until DMSO is less than 0.5%. After cells were cultured at CO₂ level of 5% and 37° C. for 24 hours, culture medium was replaced. When cell density reached 80-90%, cells underwent a succession in Trypsin-EDTA. Subsequently, culture medium was replaced every 2 or 3 days.

### MTT Assay

Medium preparation: MTT solution (5 mg/mL) was diluted to 0.5 mg/mL with cell medium.

4.5 mL/75 cm² of the Fibronectin/BSA/Collagen solution was added in 96-well plates and incubated at CO₂ level of 5% and 37° C. for 24 hours. Cells were seeded at a density of 6,000 cells/well and incubated overnight. Then cell medium was replaced by medium containing an active agent. After cells were incubated with medium containing an active agent at 37° C. for 24 hours, the medium was replaced by medium containing 0.5 mg/mL MTT (100 μg/well). After cells were incubated with medium containing MTT at 37° C. for 24 hours, the medium was removed and DMSO (100 μg/well) was added to dissolve violet crystals. The solution was centrifuged at 450 rpm for 20-30 minutes. Cell viability was calculated by OD₅₇₀.

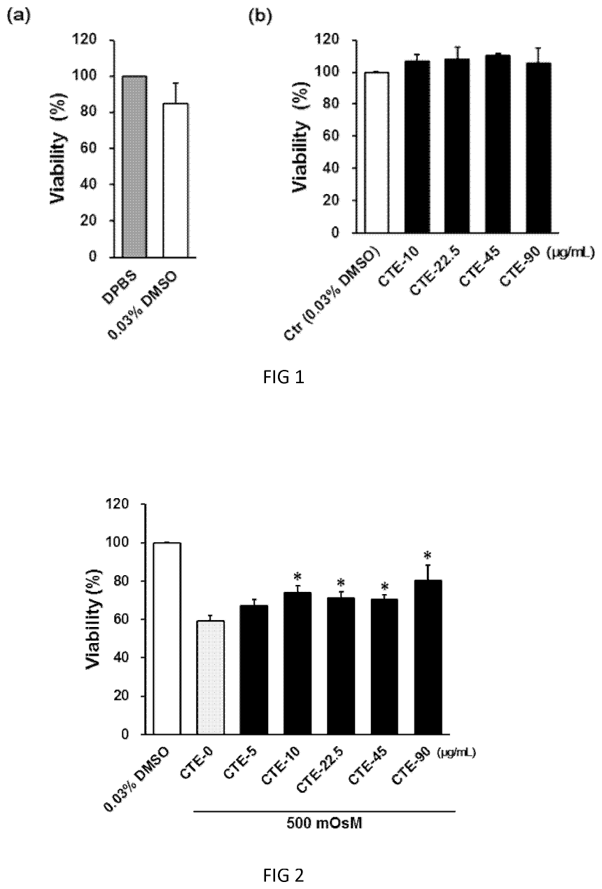

Cistanche tubulosa extract (code name: ST01) was dissolved to form the following groups: CTE-0 (ST01: 0 μg/mL; DMSO: 0.03%), CTE-10 (ST01: 10 μg/mL; DMSO: 0.03%), CTE-22.5 (ST01: 22.5 μg/mL; DMSO: 0.03%), CTE-45 (ST01: 45 μg/mL; DMSO: 0.03%) and CTE-90 (ST01: 90 μg/mL; DMSO: 0.03%) to test cell viability. Comparing with blank (DPBS), control (0.03% DMSO) does not result in significant cytotoxicity (see FIG. 1 (a)). Comparing with control (0.03% DMSO), ST01 at concentrations of 10, 22.5, 45, and 90 μg/mL does not result in cytotoxicity (see FIG. 1 (b)).

Further, cells were treated with hyperosmotic medium (500 mOsM) to test cell viability. According to results of FIG. 2, in hyperosmolarity state (500 mOsM), cell viability of corneal epithelial cells dropped to 59% in control group (0.03% DMSO) (CTE-0 group), 67% in CTE-5 group, 74% in CTE-10 group, 71% in CTE-22.5 group, 71% in CTE-45 group and 81% in CTE-90 group. In view of the results, Cistanche tubulosa extract protects corneal epithelial cells from damages induced by hyperosmolarity, which is known as a critical mechanism in dry eye.

### Example 3: Measurement of TNF-α and IFN-γ Levels

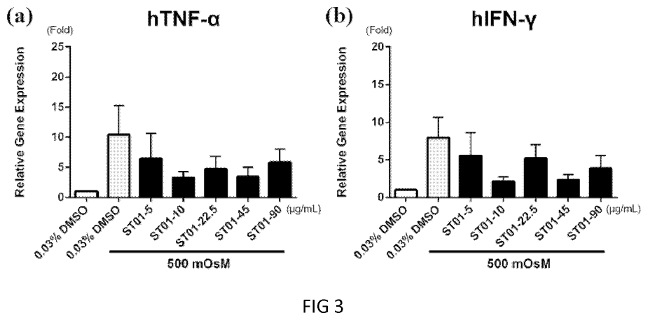

4.5 mL/75 cm² of the Fibronectin/BSA/Collagen solution was added in 6-well plates and incubated at CO₂ level of 5% and 37° C. for 24 hours. Cells were seeded at a density of 7×10⁵ cells/well and incubated overnight. Then cell medium was replaced by hyperosmotic mediums (500 mOsM) containing Cistanche tubulosa extracts: ST01-5 (ST01: 5 μg/mL; DMSO: 0.03%), ST01-10 (ST01: 10 μg/mL; DMSO: 0.03%), ST01-22.5 (ST01: 22.5 μg/mL; DMSO: 0.03%), ST01-45 (ST01: 45 μg/mL; DMSO: 0.03%) and ST01-90 (ST01: 90 μg/mL; DMSO: 0.03%). The mediums were incubated at 37° C. for 24 hours. Cells treated with Trypsin and the mediums were centrifuged at 3,000 rpm for 10 minutes to obtain cells for RNA extraction. Human Tumor Necrosis Factor-α (hTNF-α) and human Interferon-γ (hIFN-γ) were measured by real-time PCR. As shown in FIGS. 3 (a) and (b), ST01 (5-90 μg/mL) exhibited expressions of TNF-α and IFN-γ induced by hyperosmolarity.

### Example 4: Tear Film Analysis

Tear film was analyzed in subjects suffering from dry eye prior to and after taking Cistanche tubulosa extract and/or one or more dietary supplements, including docosahexaenoic acid, eicosapentanoic acid, lactoferrin, linolenic acid, lutein, maqui berry extract, phosphatidylethanolamine, phosphatidylserine, phytosterols, sphingomyelin, turmeric extract, vitamin B6, vitamin C and vitamin E. Before subjects started to take Cistanche tubulosa extract and/or one or more dietary supplements (Week 0), they discontinued medical treatments or dietary supplements for dry eye, if any, for a one-week interval, and subsequently tear film was evaluated by SBM Sistemi Ocular Surface Analyzer (O.S.A) as baseline. From Week 1 to Week 4, subjects took 225 mg Cistanche tubulosa extract once daily for up to 30 days. Tear film was evaluated by O.S.A on Day 2, 3, 4 and 5 of Week 1. From Week 2 to Week 5, O.S.A measurements were conducted once a week.

### Example 5: Dry Eye Questionnaire

Dry eye questionnaire scores assessing symptoms of ocular irritation consistent with dry eye syndrome and impacts on vision-related function were recorded for subjects suffering from dry eye prior to (baseline) and after taking Cistanche tubulosa extract and/or one or more dietary supplements, including docosahexaenoic acid, eicosapentanoic acid, lactoferrin, linolenic acid, lutein, maqui berry extract, phosphatidylethanolamine, phosphatidylserine, phytosterols, sphingomyelin, turmeric extract, vitamin B6, vitamin C and vitamin E. A paired t test was used to compare the pre and post-administration questionnaire scores. P<0.05 was considered significant. All subjects reported an improvement after taking Cistanche tubulosa extract and/or one or more dietary supplements. From available dry eye questionnaires, there were improvements in symptoms of dry eye.

### Example 6: Cistanche tubulosa Extract and Dietary Supplements

The exemplified compositions are as follows:

Composition 1: lactose, Cistanche cistanche extract, gelatin, titanium dioxide, sodium lauryl sulfate, glycerin, maqui berry extract, magnesium stearate, sodium aluminosilicate, and vitamin B6.

Composition 2: lutein, safflower oil, vitamin E, gelatin, docosahexaenoic acid, glycerin, lecithin, and titanium dioxide.

All publications, patents and patent applications cited in this specification are incorporated herein by reference in their entireties. While the foregoing has been described in terms of various embodiments, the skilled artisan will appreciate that various modifications, substitutions, omissions, and changes may be made without departing from the spirit thereof.

What is claimed is:

1. A method for relieving dry eye syndrome comprising administering to a subject in need thereof an effective amount of Cistanche tubulosa extract.

2. The method of claim 1, wherein dry eye syndrome is associated with smoke, particulates, dry air, air conditioning, prolonged use of electronic devices, contact lens wear, aging, hormone changes, inflammation, autoimmune diseases, refractive surgery or medication.

3. The method according to claim 1, wherein the extract comprises echinacocide, aceteoside, isoacteoside, and tubuloside A.

4. The method of claim 1, wherein Cistanche tubulosa extract is administered orally in a pharmaceutically acceptable formulation selected from the group consisting of a pill, tablet, capsule, powder, granule, non-aqueous liquid, aqueous liquid, suspension, solution, emulsion, and lyophilized formulation.

5. The method of claim 1, wherein Cistanche tubulosa extract is administered once daily in an amount from 50 to 250 mg.

6. The method of claim 1, wherein Cistanche tubulosa extract is administered in combination with one or more additional dietary supplements selected from a group consisting of docosahexaenoic acid, eicosapentanoic acid, lactoferrin, linolenic acid, lutein, maqui berry extract, phosphatidylethanolamine, phosphatidylserine, phytosterols, sphingomyelin, turmeric extract, vitamin B6, vitamin C and vitamin E.

7. The method of claim 1, wherein dry eye syndrome is relieved by increasing tear production.

8. The method of claim 1, wherein dry eye syndrome is relieved by improving meibomian gland dysfunction.

9. The method of claim 1, wherein dry eye syndrome is relieved by increasing tear film break-up time or tear film lipid layer thickness.

10. A method for relieving dry eye comprising administering a dietary supplement to a subject in need thereof, wherein the dietary supplement comprising an effective amount of Cistanche tubulosa extract.
